(19) United States
(12) Patent Application Publication
(10) Pub. No.: US 2020/0031771 A1
(43) Pub. Date: Jan. 30, 2020

(54) SUBSTITUTED PHENYLPROPIONIC ACID ENANTIOMER AND MANUFACTURING METHOD, COMPOSITION, AND APPLICATION OF SAME

(71) Applicant: SHENZHEN CHIPSCREEN BIOSCIENCES CO., LTD., Shenzhen, Guangdong (CN)

(72) Inventors: Jindi YU, Shenzhen (CN); Desi PAN, Shenzhen (CN); Song SHAN, Shenzhen (CN); Zhibin LI, Shenzhen (CN); Xianping LU, Shenzhen (CN)

(73) Assignee: Shenzhen Chipscreen Biosciences Co., Ltd., Shenzhen, Guangdong (CN)

(21) Appl. No.: 16/337,394

(22) PCT Filed: Sep. 27, 2017
(86) PCT No.: PCT/CN2017/103619

§ 371 Date: Mar. 27, 2019

(30) Foreign Application Priority Data
Sep. 27, 2016 (CN) 201610856914.7

(51) Int. Cl. C07D 209/86; A61P 3/08

(52) U.S. Cl. CPC C07D 209/86; A61P 3/08

(57) ABSTRACT

The present invention discloses an enantiomeric compound (−)-2-[(2-(4-fluorobenzoyl)phenyl)amine]-3-[(4-(2-carbazole-ethoxy)phenyl)]propionic acid as represented by formula (I), or a pharmaceutical salt thereof, and a manufacturing method of the compound and application of same. The enantiomeric compound demonstrates relatively better activation of RXR/PPAR-, RXR/PPAR- and RXR/PPAR-heterodimer expression and sugar reduction in a db/db mouse model compared to a (+)-enantiomer.

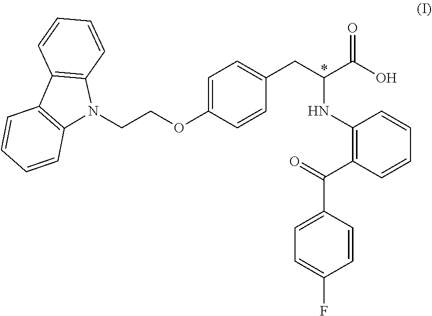 [SMILES: O=C(c1ccc(F)cc1)c1ccccc1NC(Cc1ccc(OCCn2c3ccccc3c3ccccc32)cc1)C(=O)O]

### CROSS REFERENCE TO RELATED APPLICATIONS

This application claims the priority of Chinese Patent Application No. 201610856914.7, filed on Sep. 27, 2016, and titled with “(−)-2-[(2-(4-FLUOROBENZOYL)PHENYL)AMINO]-3-[(4-(2-CARBAZOLE-ETHOXY)PHENYL)]PROPIONIC ACID, MANUFACTURING METHOD, COMPOSITION AND APPLICATION OF SAME”, and the disclosures of which are hereby incorporated by reference.

### FIELD

The present invention relates to the field of pharmaceutical technology, specifically to the (−)-enantiomer of 2-[(2-(4-fluorobenzoyl)phenyl)amino]-3-[(4-(2-carbazole-ethoxy) phenyl)]propionic acid and pharmaceutically acceptable salt thereof, preparation method thereof, composition containing the same, and application thereof.

### BACKGROUND

PPARγ is a member of nuclear receptor superfamily that is primarily expressed in adipose tissue. PPARα, another member of this family, is expressed primarily in liver tissue and can be activated by a class of ligands called fibrates and reduce the levels of triglyceride and cholesterol. PPARα can stimulate the proliferation of peroxidase and accelerates the oxidation of fatty acids, thereby reducing the fatty acid content in the blood (Keller and Wahli: Trends Endocrin Metab 1993, 4:291-296). It has recently been reported that PPARδ acts as a regulator of lipid metabolism with extensive fat-“burning” effects. In in vitro experiments, activation of PPARδ in adipose and skeletal muscle cells contributes to the oxidation and utilization of fatty acids. In animal adipose tissue with low PPARα expression, activation of PPARδ can specifically induce expression of genes involved in fatty acid oxidation and energy expenditure, thereby improving lipid content and reducing weight. More importantly, these animals with activated PPARδ were completely resistant to high-fat diets as well as genetic factors (Lepr (db/db))-induced obesity. Short-term treatment of Lepr (db/db) mice with PPARδ activator can consume accumulated fat, whereas treating PPARδ-deficient mice with a high-fat diet can induce obesity (Wang Y X et al., Cell 2003 Apr. 18; 113 (2): 159-70).

PPARα, PPARγ, and PPARδ can each form heterodimers with the retinoic acid X receptor. Therefore, RXR/PPAR heterodimers also play an important role in regulating the balance of cellular sugar and lipid and adipocyte differentiation. It has been reported that some novel compounds including activators of PPARγ and dual activators of PPARα/PPARγ have a good effect in preventing and treating metabolic syndrome in humans and animals (WO 00/08002, WO01/57001 A1, U.S. Pat. No. 6,054,453, EP088317 B1, WO97/25042, WO02/26729 A2 and U.S. Pat. No. 6,353,018 B1). Therefore, screening for new compounds with PPARα, PPARγ and PPARδ activator properties is of great importance for the comprehensive treatment of metabolic disorders. These metabolic disorders include metabolic syndromes such as diabetes, hypertension, obesity, insulin resistance, hypertriglyceridemia, hyperglycemia, high cholesterol, arteriosclerosis, coronary heart disease, and other cardiovascular diseases.

The applicant discloses a class of aralkyl amino acid activators of PPARα, γ and δ in Chinese patent CN1257893C, that have excellent hypoglycemic and lipid-lowering activity, wherein example 15 specifically discloses 2-[(2-(4-fluorobenzoyl)phenyl)amino]-3-[(4-(2-carbazole-ethoxy)phenyl)]propionic acid and the preparation method thereof and examples 21-28 disclose the pharmacological activity of the compound. 2-[(2-(4-fluorobenzoyl)phenyl)amino]-3-[(4-(2-carbazole-ethoxy)phenyl)]propionic acid is a racemate, whereas Chinese patent CN1257893 does not disclose its (+) or (−) enantiomer, nor the preparation method and pharmacological activity thereof.

Generally, a compound will have multiple pairs of enantiomers that are mirror images of each other based on the difference in chiral position. Except for optical activity, the two enantiomers mirrored to each other generally have the same physical properties. In particular, the two enantiomers are generally completely or nearly identical in melting point, boiling point, solubility, density and refractive rate, but are completely opposite in terms of optical rotation. Since the two enantiomers rotated the plane of polarized light to the same extent, but in opposite directions, no net optical rotation will be observed when they are mixed. In other words, the optical rotation of the racemate is theoretically zero, and practically close to zero. Due to differences in optical activity, racemates and individual enantiomers may differ in their physiological activity and toxicity. However, this difference in physiological activity and toxicity is usually unpredictable, so it is not possible to predict from the difference in optical rotation in advance. In general, in most cases, the physiological activities (including pharmacodynamic effects) of the two isomers and the physicochemical properties other than the optical activity are identical. In rare cases, there exit certain pharmacodynamic differences. From the point of view of the difference in pharmacodynamics, there are mainly the following types of conditions. 1) The enantiomers have similar pharmaceutical property, but the activity intensity is different. For example, the antibacterial activity of ofloxacin S-isomer is 8-135 times stronger than the R-isomer (Mitscher L A et al., J Med Chem, 1987, 30(12): 2283-6). 2) The enantiomers are opposite in pharmaceutical property and mutually antagonistic. For example, the (+)-isomer of Picenadol is an opioid receptor agonist and the (−)-isomer is an opioid receptor antagonist (Carter R B et al., J Pharmacol Exp Ther, 1985, 234 (2): 299-306). 3) The enantiomers are complementary in pharmaceutical property to each other. For example, the R isomer of the diuretic Indacrinone has a diuretic effect, as well as a side effect of increasing uric acid in the blood, while the S isomer promotes the excretion of uric acid and can effectively reduce the side effect of the R isomer. Therefore, it is beneficial to use them in combination. Further studies have shown that the therapeutic effect is the best when the ratio of S to R isomer is 1:4 or 1:8 (Tobert J. A. et al., Clin Pharmacol Ther, 1981, 29(3): 344-50). 4) Different enantiomers have different pharmacological activities. For example, the antihypertensive drug labetalol has two chiral centers, wherein the (R,R)-isomer is a non-selective β-adrenoceptor antagonist, the (S,R)-isomer is an al-adrenoceptor antagonist, the (S,S)-isomer has weak al-adrenoceptor blockade, while the (R,S)-isomer is inactive (Pieter A. et al., Drugs, 1993, 45(4): 509-517). 5) One of the enantiomers has strong toxic side effects. For example, thalidomide has been used as a sedative to treat pregnancy reactions, but caused thousands of cases of teratogenicity after taken by pregnant women, because the S-isomer not only has sedative effects but also has strong embryotoxicity and teratogenic effects (Blaschke G. et al., Arzneim-Forsch, 1979, 29(10), 1640-2). Therefore, it is necessary to resolve the pure enantiomers from the racemic mixture and study their properties, as the physiological differences between the enantiomers and the racemic mixture are unpredictable.

The inventors prepared the (−)-enantiomer (levoisomerr) of 2-[(2-(4-fluorobenzoyl)phenyl)amino]-3-[(4-(2-carbazole-ethoxy)phenyl)]propionic acid and studied the pharmacological activity thereof.

### SUMMARY

The inventors of the present application attempted to prepare and obtained each of the enantiomers of 2-[(2-(4-fluorobenzoyl)phenyl)amino]-3-[(4-(2-carbazole-ethoxy)phenyl)]propionic acid and studied their pharmacological activities. It was unexpectedly found that the two isomers have significantly different pharmacological effects, wherein the levoisomer ((−)-enantiomer at a specific chiral position (see the position indicated by “*” in formula I) is significantly superior to (+)-enantiomer (ie. dextroisomer) in terms of activating the expression of RXR/PPAR-, RXR/PPAR- and RXR/PPAR-heterodimers and the hypoglycemic effect demonstrated in the db/db mouse model.

Based on the finding, in a first aspect, the present disclosure provides 2-[(2-(4-fluorobenzoyl)phenyl)amino]-3-[(4-(2-carbazole-ethoxy)phenyl)]propionic acid or a pharmaceutically acceptable salt thereof that can selectively activate PPAR-α, PPAR-γ and PPAR-6.

In a second aspect, the present disclosure provides a preparation method of (−)-2-[(2-(4-fluorobenzoyl)phenyl)amino]-3-[(4-(2-carbazole-ethoxy)phenyl)]propionic acid or a pharmaceutically acceptable salt thereof.

In a third aspect, the present disclosure provides use of (−)-2-[(2-(4-fluorobenzoyl)phenyl)amino]-3-[(4-(2-carbazole-ethoxy)phenyl)]propionic acid or a pharmaceutically acceptable salt thereof in the preparation of a medicament for treating a disease associated with metabolic syndrome such as diabetes.

The chemical structure of the compound (−)-2-[(2-(4-fluorobenzoyl)phenyl)amino]-3-[(4-(2-carbazole-ethoxy)phenyl)]propionic acid according to the present disclosure is shown in formula (I):

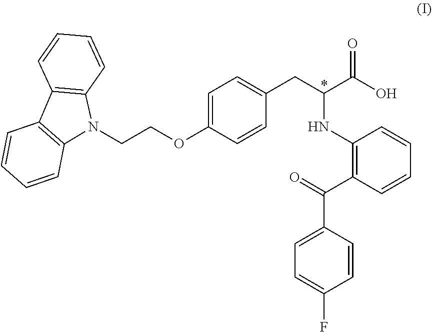 [SMILES: O=C(c1ccc(F)cc1)c1ccccc1NC(Cc1ccc(OCCn2c3ccccc3c3ccccc32)cc1)C(=O)O]

the position indicated by * a chiral position

Enantiomer 1: (−)-2-[(2-(4-fluorobenzoyl)phenyl)amino]-3-[(4-(2-carbazole-ethoxy)phenyl)]propionic acid;

Enantiomer 2: (+)-2-[(2-(4-fluorobenzoyl)phenyl)amino]-3-[(4-(2-carbazole-ethoxy)phenyl)]propionic acid

“Pharmaceutically acceptable salt” as used herein means a salt that is pharmaceutically acceptable. Bases used in the preparation of the salts include, but are not limited to, alkali metal (eg, sodium, potassium) hydroxides, alkaline earth metal (eg, calcium, magnesium) hydroxides, aqueous ammonia, and organic amines (NR¹R²R³, wherein R¹, R² and R³ may be the same or different and preferably being hydrogen atom or a C1-C4 alkyl group).

“Pharmaceutically acceptable” as used herein is understood to be suitable for human and animal use within a reasonable medical range without excessive side effects including toxicity, allergic reactions, irritation and complications, and other deleterious effects.

The present invention also relates to a pharmaceutical composition comprising (−)-2-[(2-(4-fluorobenzoyl)phenyl)amino]-3-[(4-(2-carbazole-ethoxy)phenyl)]propionic acid or a pharmaceutically acceptable salt thereof, a pharmaceutical adjuvant including a pharmaceutically acceptable excipient and/or carrier. The (−)-2-[(2-(4-fluorobenzoyl)phenyl)amino]-3-[(4-(2-carbazole-ethoxy)phenyl)]propionic acid or a pharmaceutically acceptable salt thereof in the present disclosure may be used in combination with one or more other active pharmaceutical ingredients, which may be in any pharmaceutically acceptable combination.

The term “pharmaceutically acceptable excipient and/or carrier” as used herein means a non-toxic, inert, solid, semi-solid or liquid filler, diluent, encapsulating material or formulation aid of any type. Remington's Pharmaceutical Sciences Ed. by Gennaro, Mack Publishing, Easton, Pa., 1995, discloses various carriers for formulating pharmaceutical compositions and known techniques for preparing these pharmaceutical compositions. Some examples of materials that can be used as pharmaceutically acceptable carriers include, but are not limited to, sugars such as lactose, glucose, and sucrose; starches such as corn starch and potato starch; cellulose and its derivatives, such as sodium carboxymethylcellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; diols such as propylene glycol; esters such as ethyl oleate and ethyl laurate; agar; detergents such as TWEEN™ 80; buffers such as magnesium hydroxide and aluminum hydroxide; alginic acid; pyrogen-free water; isotonic saline; Ringer's solution; ethanol; and phosphate buffer solution and other non-toxic compatible lubricants, such as sodium lauryl sulfate and magnesium stearate; and colorants, coating agents, sweeteners, flavorings, and fragrances; preservatives and antioxidants may also be present in the compositions, as judged by the formulator. If filtration or other terminal sterilization processes are not feasible, the formulation can be produced under sterile conditions.

The (−)-2-[(2-(4-fluorobenzoyl)phenyl)amino]-3-[(4-(2-carbazole-ethoxy)phenyl)]propionic acid or a pharmaceutically acceptable salt thereof as described in the present invention can be prepared into various dosage forms with conventional pharmaceutical excipients, such as but not limited to oral preparations (tablets, capsules, powders, granules, syrups, pills, etc.), injection preparations, and topical preparations, etc. The pharmaceutical composition of the present invention may usually contain 0.5 to 70% by weight of the active ingredient, preferably 1 to 20% by weight.

### Advantageous Effects of the Present Disclosure

In the present invention, (−)-2-[(2-(4-fluorobenzoyl)phenyl)amino]-3-[(4-(2-carbazole-ethoxy)phenyl)]propionic acid is separated from the 2-[(2-(4-fluorobenzoyl)phenyl) amine]-3-[(4-(2-carbazole-ethoxy)phenyl)]propionic acid enantiomers in the racemate, which activates the expression of RXR/PPAR-, RXR/PPAR- and RXR/PPAR-heterodimers and exhibits a significantly better hypoglycemic effect in the db/db mouse model, further improving the therapeutic effect of [(2-(4-fluorobenzoyl)phenyl)amine]-3-[(4-(2-carbazole-ethoxy)phenyl)]propionic acid compounds on RXR or PPAR nuclear receptors associated diseases including type 2 diabetes, lipid metabolism disorders, syndrome X, cardiovascular disease, coronary artery disease, hypercholesterolemia and obesity.

### BRIEF DESCRIPTION OF DRAWINGS

FIG. 1 shows the activation activity of enantiomers 1 and 2 of the present disclosure on PPARα in an in vitro reporter assay experimental model;

FIG. 2 shows the activation activity of enantiomers 1 and 2 of the present disclosure on PPARγ in an in vitro reporter assay experimental model;

FIG. 3 shows the activation activity of enantiomers 1 and 2 of the present disclosure on PPAR-6 in an in vitro reporter assay experimental model.

### DETAILED DESCRIPTION

The contents of the present disclosure are further described below with reference to examples, but the protection scope of the present disclosure is not limited to these examples. The percentages stated in the present disclosure are all percentages by weight unless otherwise specified. The range of values, such as units of measure or percentages, described in the specification are intended to provide an unambiguous written reference. The person skilled in the art will still be able to obtain the desired results based on the teachings and principles of the present disclosure, using temperatures, concentrations, amounts, etc. outside of this range or different from a single value.

### Example 1: Preparation of Enantiomer 1 and 2 of [(2-(4-fluorobenzoyl)phenyl)amine]-3-[(4-(2-carbazole-ethoxy)phenyl)]propionic acid

20 g of mixture of enantiomers was dissolved in 2070 ml of methanol and applied to a supercritical chromatograph Chiralpak column (AY 20*250 mm). The column was first washed with a methanol/carbon dioxide mixture (volume ratio of 4:1) at a flow rate of 80 mL/min for 120 minutes, and then eluted with a methanol/carbon dioxide/aqueous ammonia mixture (volume ratio of 6:4:0.005) at a flow rate of 80 mL/min for 30 minutes. After about 6 minutes, an elution peak of the objective compound enantiomer 1 was obtained; after about 11 minutes, a peak of the enantiomer 2 was obtained. After evaporation of the solvent, enantiomer 1 (15 g) and enantiomer 2 (2 g) were obtained, respectively.

### (−)-2-[(2-(4-fluorobenzoyl)phenyl)amino]-3-[(4-(2-carbazole-ethoxy)phenyl)]propionic acid (Enantiomer 1)

content=100%

ee %=100%

[α]²⁴_{D}=−111.5 (c=0.01, DMSO)

Infrared spectrum (KBr, cm⁻¹): 3323, 3047, 2983, 2938, 2870, 2730, 2492, 1922, 1893, 1622, 1598, 1508, 1456, 1387, 1249, 927, 850, 751;

¹H-NMR (DMSO-d₆) δ 2.88 (dd, 1H, J=13.8, 6.1 Hz, one of CH₂), 3.06 (dd, 1H, J=13.8, 5 Hz, one of CH₂), 4.01-4.04 (m, H, CH), 4.25 (t, J=5.3 Hz, 2H, CH₂), 4.72 (t, J=5.3 Hz, 2H, CH₂), 6.44 (t, 1H, J=7.5 Hz, Ar—H), 6.60 (d, J=8.7 Hz, 2H, Ar—H), 6.70 (d, J=8.6 Hz, 1H, Ar—H), 7.00 (d, J=8.6 Hz, 2H, Ar—H), 7.19 (t, 2H, J=7.5 Hz, Ar—H), 7.24 (dd, 1H, J=8.1, 1.5 Hz, Ar—H), 7.26-7.32 (m, 3H, Ar—H), 7.43 (td, 2H, J=7.7, 1 Hz, Ar—H), 7.55-7.58 (m, 2H, Ar—H), 7.63 (d, J=8.3 Hz, 2H, Ar—H), 8.13 (d, J=7.8 Hz, 2H, Ar—H), 8.64 (d, J=7.0 Hz, 1H, NH);

HRMS (C₃₆H₂₉FN₂O₄) calcd. (%): 572.2111; found (%): 572.2108.

### (+)-2-[(2-(4-fluorobenzoyl)phenyl)amino]-3-[(4-(2-carbazole-ethoxy)phenyl)]propionic acid (Enantiomer 2)

content=100%

ee %=99.5%

[α]²⁴_{D}=98.5 (c=0.01, DMSO)

Infrared spectrum (KBr, cm⁻¹): 3318, 3048, 2927, 2869, 2495, 1921, 1892, 1621, 1598, 1509, 1457, 1250, 1154, 927, 849, 751;

¹H-NMR (DMSO-d₆) δ 2.91 (dd, 1H, J=13.8, 6.2 Hz, one of CH₂), 3.06 (dd, 1H, J=13.8, 5 Hz, one of CH₂), 4.14-4.17 (m, 1H, CH), 4.24 (t, J=5.3 Hz, 2H, CH₂), 4.72 (t, J=5.3 Hz, 2H, CH₂), 6.47 (t, 1H, J=7.4 Hz, Ar—H), 6.61 (d, J=8.6 Hz, 2H, Ar—H), 6.73 (d, J=8.6 Hz, 1H, Ar—H), 7.01 (d, J=8.6 Hz, 2H, Ar—H), 7.18 (t, 2H, J=7.4 Hz, Ar—H), 7.26 (dd, 1H, J=8.0, 1.4 Hz, Ar—H), 7.29-7.32 (m, 3H, Ar—H), 7.42 (td, 2H, J=7.7, 0.9 Hz, Ar—H), 7.56-7.59 (m, 2H, Ar—H), 7.63 (d, J=8.3 Hz, 2H, Ar—H), 8.12 (d, J=7.7 Hz, 2H, Ar—H), 8.63 (d, J=7.1 Hz, 1H, NH);

HRMS (C₃₆H₂₉FN₂O₄) calcd. (%): 572.2111; found (%): 572.2109.

### Example 2: Preparation of Sodium (−)-2-[(2-(4-fluorobenzoyl)phenyl)amino]-3-[(4-(2-carbazole-ethoxy)phenyl)]propionate (Sodium Salt of Enantiomer 1)

30 ml of tetrahydrofuran was added into a 50 mL round bottom flask, and (−)-2-[(2-(4-fluorobenzoyl)phenyl)amino]-3-[(4-(2-carbazole-ethoxy)phenyl)]propionic acid (1000 mg, 1.75 mmol) was added with stirring, until dissolved. Sodium hydroxide (71 mg, 1.77 mmol) was dissolved in 2 mL of methanol. Then, the methanol solution of sodium hydroxide was added dropwise into (−)-2-[(2-(4-fluorobenzoyl)phenyl)amino]-3-[(4-(2-carbazole-ethoxy)phenyl)]propionic acid in tetrahydrofuran under stirring and the reaction was continued with stirring for 30 minutes. The mixture was distilled under reduced pressure and the distillation residue was dissolved in 2 mL of dichloromethane. 30 ml of isopropyl ether was added into a 50 ml round bottom flask and the above dichloromethane solution was added dropwise with stirring. After the completion of the dropwise addition, the mixture was stirred for 2 minutes and filtered off with suction. The filter cake was rinsed with 5 ml of isopropyl ether, dried under vacuum at room temperature for 4 hours, and dried under vacuum at 60° C. to a loss on drying less than 0.5% by weight to give a yellow solid sodium (−)-2-[(2-(4-fluorobenzoyl)phenyl)amino]-3-[(4-(2-carbazole-ethoxy)phenyl)]propionate (1 g, 1.68 mmol, 96.3% yield).

content=98.7%

ee %=96.1%

[α]²⁴_{D}=−119.5 (c=0.01, DMSO)

Infrared spectrum (KBr, cm¹): 3352, 3051, 2969, 2930, 2871, 1921, 1891, 1617, 1598, 1509, 1457, 1400, 1242, 928, 850, 750;

X-Ray Powder Diffraction: Amorphous

The measured data and analytical results of NMR (Bruker AVANCE III HD 500, DMSO-d₆), including 1-H NMR, 13-C NMR, COSY, HMQC and HMBC are shown in Table 1:

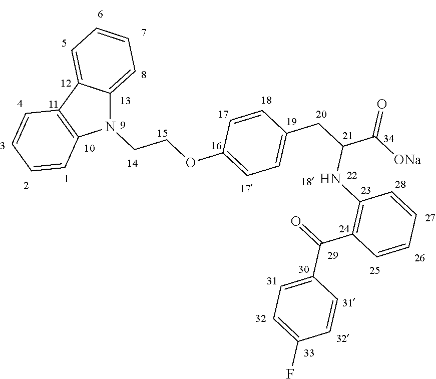

TABLE 1
the data and analysis of NMR spectra of sodium
(−)-2-[(2-(4-fluorobenzoyl)phenyl)amino]-3-[(4-(2-carbazole-ethoxy)phenyl)]propionate

| | | | | HSQC | HMBC |
|---|---|---|---|---|---|
| Atomic | Chemical shift | Chemical shift | COSY | Homo | Hetero |
| number | δH (ppm) | δC (ppm) | 1H-1H | 13C-1H | 13C-1H |
| 1 (8) | 7.63 (d, 2H, J = 8.3 Hz) | 109.56 | 7.63 | 109.56 (7.63) | 109.56 |
| | | | (7.41-7.45) | | (7.41-7.45, |
| | | | 7.63 | | 7.17-7.19, 8.13) |
| | | | (7.17-7.19) | | |
| 2 (7) | 7.41-7.45 (m, 2H) | 125.6 | 7.41-7.45 | 125.60 | 125.60 (8.13, |
| | | | (7.17-7.19) | (7.41-7.45) | 7.17-7.19) |
| | | | 7.41-7.45 | | |
| | | | (7.63) | | |
| | | | 7.41-7.45 | | |
| | | | (8.13) | | |
| 3 (6) | 7.17-7.19 (m, 2H) | 118.83 | 7.17-7.19 | 118.83 | 118.83 (7.63) |
| | | | (7.41-7.45) | (7.17-7.19) | |
| | | | 7.17-7.19 | | |
| | | | (8.13) | | |
| | | | 7.17-7.19 | | |
| | | | (7.63) | | |
| 4 (5) | 8.13 (d, 2H, J = 7.7 Hz) | 120.09 | 8.13 | 120.09 (8.13) | 120.09 |
| | | | (7.17-7.19) | | (7.41-7.45) |
| | | | 8.13 | | |
| | | | (7.41-7.45) | | |
| 10 (13) | | 140.21 | | | 140.21 (4.73, |
| | | | | | 7.41-7.45, 8.13) |
| 11 (12) | | 122.1 | | | 122.10 |
| | | | | | (7.17-7.19, 7.63, |
| | | | | | 8.13) |
| 14 | 4.73 (t, 2H, J = 5.3 Hz) | 42.08 | 4.73 (4.24) | 42.08 (4.73) | 42.08 (4.24) |
| 15 | 4.24 (t, 2H, J = 5.4 Hz) | 66.17 | 4.24 (4.73) | 66.17 (4.24) | 66.17 (4.73) |
| 16 | | 156.23 | | | 156.23 (7.01, |
| | | | | | 6.59, 4.23) |
| 17 (17′) | 6.59 (d, 2H, J = 8.7 Hz) | 113.64 | 6.59 (7.01) | 113.64 (6.59) | 113.64 (7.01) |
| 18 (18′) | 7.01 (d, 2H, J = 8.6 Hz) | 130.35 | 7.01 (6.59) | 130.35 (7.01) | 130.35 (2.88, |
| | | | | | 3.06, 6.59) |
| 19 | | 131.51 | | | 131.51 |
| | | | | | (2.88, 3.06, |
| | | | | | 3.90-3.93, 6.59) |
| 20 | 2.88 (dd, 1H, J = 13.7, | 37.13 | 2.88 | 37.13 (2.88, | 37.13 |
| | 5.9 Hz) | | (3.90-3.93) | 3.06) | (3.90-3.93, 7.01) |
| | 3.06 (dd, 1H, J = 13.7, | | 3.06 | | |
| | 5.1 Hz) | | (3.90-3.93) | | |
| 21 | 3.90-3.93 (m, 1H) | 59 | 3.90-3.93 | 59 | 59.00 (2.88, |
| | | | (2.88) | (3.90-3.93) | 3.06, 8.74) |
| | | | 3.90-3.93 | | |
| | | | (3.06) | | |
| | | | 3.90-3.93 | | |
| | | | (8.74) | | |
| 22 | 8.74 (d, 1H, J = 6.7 Hz) | | 8.74 | | |
| | | | (3.90-3.93) | | |
| 23 | | 150.31 | | | 150.31 |
| | | | | | (7.20-7.26, |
| | | | | | 3.90-3.93) |
| 24 | | 116.5 | | | 116.50 (6.39, |
| | | | | | 6.66, 8.74) |
| 25 | 7.20-7.26 (m, 1H) | 134.22 | 7.20-7.26 | 134.22 | 134.22 |
| | | | (6.39) | (7.20-2.26) | (7.20-7.26) |
| 26 | 6.39 (t, 1H, J = 7.3 Hz) | 112.48 | 6.39 | 112.48 (6.39) | 112.48 (6.66) |
| | | | (7.20-7.26) | | |
| | | | 6.39 | | |
| | | | (7.20-7.26) | | |
| 27 | 7.20-7.26 (m, 1H) | 134.48 | 7.20-7.26 | 134.48 | 134.48 |
| | | | (6.39) | (7.20-7.26) | (7.20-7.26, 6.39) |
| | | | 7.20-7.26 | | |
| | | | (6.66) | | |
| 28 | 6.66 (d, 1H, J = 8.6 Hz) | 112.63 | 6.66 | 112.63 (6.66) | 112.63 (6.39, |
| | | | (7.20-7.26) | | 8.74) |
| 29 | | 195.72 | | | 195.72 |
| | | | | | (7.55-7.58, |
| | | | | | 7.20-7.26) |
| 30 | | 136.8 | | | 136.80 |
| | | | | | (7.28-7.32) |
| 31 (31′) | 7.55-7.58 (m, 2H) | 131.25 | 7.55-7.58 | 131.25 | |
| | | 3JF-C = 8.8 | (7.28-7.32) | (7.55-7.58) | |
| 32 (32′) | 7.28-7.32 (m, 2H) | 115.1 | 7.28-7.32 | 115.10 | 115.10 |
| | | 2JF-C = 22.5 | (7.55-7.58) | (7.28-7.32) | (7.55-7.58) |
| 33 | | 163.4 | | | 163.40 |
| | | 1JF-C = 248 | | | (7.55-7.58, |
| | | | | | 7.28-7.32) |
| 34 | | 173.67 | | | 173.67 (2.88, |
| | | | | | 3.06, 3.90-3.93, |
| | | | | | 8.74) |

FAB-MS (m/z): 595 (M+1)

### Example 3: Preparation of Sodium (+)-2-[(2-(4-fluorobenzoyl)phenyl)amine]-3-[(4-(2-carbazole-ethoxy)phenyl)]propionate (Sodium Salt of Enantiomer 2)

30 ml of tetrahydrofuran was added into a 50 mL round bottom flask, and (+)-2-[(2-(4-fluorobenzoyl)phenyl)amino]-3-[(4-(2-carbazole-ethoxy)phenyl)]propionic acid (1000 mg, 1.75 mmol) was added with stirring, until dissolved. Sodium hydroxide (71 mg, 1.77 mmol) was dissolved in 2 mL of methanol. Then the methanol solution of sodium hydroxide was added dropwise into (+)-2-[(2-(4-fluorobenzoyl)phenyl)amino]-3-[(4-(2-carbazole-ethoxy) phenyl)]propionic acid in tetrahydrofuran under stirring and the reaction was continued with stirring for 30 minutes. The mixture was distilled under reduced pressure and the distillation residue was dissolved in 2 mL of dichloromethane. 30 ml of isopropyl ether was added into a 50 ml round bottom flask and the above dichloromethane solution was added dropwise with stirring. After the completion of the dropwise addition, the mixture was stirred for 2 minutes and filtered off with suction. The filter cake was rinsed with 5 ml of isopropyl ether, dried under vacuum at room temperature for 4 hours, and dried under vacuum at 60° C. to a loss on drying less than 0.5% by weight to give a yellow solid sodium (+)-2-[(2-(4-fluorobenzoyl)phenyl)amino]-3-[(4-(2-carbazole-ethoxy)phenyl)]propionate (980 mg, 1.65 mmol, 94.1% yield).

content=97.3%

ee %=98.4%

[α]²⁴_{D}=128.8 (c=0.01, DMSO)

Infrared spectrum (KBr, cm⁻¹): 3354, 3051, 2969, 2929, 2871, 1922, 1891, 1617, 1598, 1509, 1401, 1243, 928, 850, 750;

X-Ray Powder Diffraction: Amorphous

The measured data and analytical results of NMR (Bruker AVANCE III HD 500, DMSO-d₆), including 1-H NMR, 13-C NMR, COSY, HMQC and HMBC are shown in Table 2:

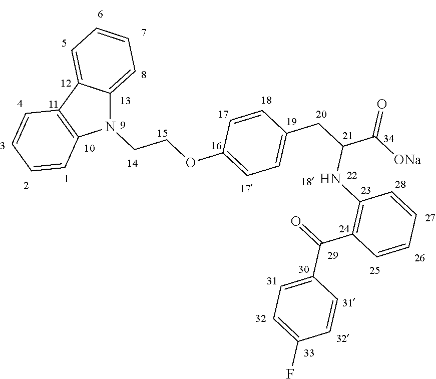

TABLE 2
the data and analysis of NMR spectra of sodium
(+)-2-[(2-(4-fluorobenzoyl)phenyl)amine]-3-[(4-(2-carbazole-ethoxy)phenyl)]propionate

| | | | | | HMBC |
|---|---|---|---|---|---|
| Atomic | Chemical shift | Chemical shift | COSY | HSQC | Hetero |
| number | δH (ppm) | δC (ppm) | 1H-1H | Homo13C-1H | 13C-1H |
| 1 (8) | 7.63 (d, 2H, | 109.55 | 7.63 | 109.55 (7.63) | 109.55 |
| | J = 8.3 Hz) | | (7.41-7.45) | | (7.41-7.45, |
| | | | 7.63 | | 7.17-7.19, 8.13) |
| | | | (7.17-7.19) | | |
| 2 (7) | 7.41-7.45 (m, | 125.59 | 7.41-7.45 | 125.59 | 125.59 (8.13, |
| | 2H) | | (7.17-7.19) | (7.41-7.45) | 7.17-7.19) |
| | | | 7.41-7.45 | | |
| | | | (7.63) | | |
| | | | 7.41-7.45 | | |
| | | | (8.13) | | |
| 3 (6) | 7.17-7.19 (m, | 118.83 | 7.17-7.19 | 118.83 | 118.83 (7.63) |
| | 2H) | | (7.41-7.45) | (7.17-7.19) | |
| | | | 7.17-7.19 | | |
| | | | (8.13) | | |
| | | | 7.17-7.19 | | |
| | | | (7.63) | | |
| 4 (5) | 8.13 (d, 2H, | 120.09 | 8.13 | 120.09 (8.13) | 120.09 |
| | J = 7.7 Hz) | | (7.17-7.19) | | (7.41-7.45) |
| | | | 8.13 | | |
| | | | (7.41-7.45) | | |
| 10 (13) | | 140.21 | | | 140.21 (4.73, |
| | | | | | 7.41-7.45, 8.13) |
| 11 (12) | | 122.1 | | | 122.10 |
| | | | | | (7.17-7.19, 7.63, |
| | | | | | 8.13) |
| 14 | 4.73 (t, 2H, | 42.07 | 4.73 | 42.07 (4.73) | 42.07 (4.24) |
| | J = 5.3 Hz) | | (4.24) | | |
| 15 | 4.24 (t, 2H, | 66.17 | 4.24 | 66.17 (4.24) | 66.17 (4.73) |
| | J = 5.4 Hz) | | (4.73) | | |
| 16 | | 156.23 | | | 156.23 (7.01, |
| | | | | | 6.59, 4.24) |
| 17 (17′) | 6.59 (d, 2H, | 113.64 | 6.59 | 113.64 (6.59) | 113.64 (7.01) |
| | J = 8.7 Hz) | | (7.01) | | |
| 18 (18′) | 7.01 (d, 2H, | 130.35 | 7.01 | 130.35 (7.01) | 130.35 (2.88, |
| | J = 8.7 Hz) | | (6.59) | | 3.06, 6.59) |
| 19 | | 131.5 | | | 131.50 |
| | | | | | (2.88, 3.06, |
| | | | | | 3.90-3.93, 6.59) |
| 20 | 2.88 (dd, 1H, | 37.13 | 2.88 | 37.13 (2.88, | 37.13 |
| | J = 13.7, 5.9 Hz) | | (3.90-3.93) | 3.06) | (3.90-3.93, 7.01) |
| | 3.07 (dd, 1H, | | 3.06 | | |
| | J = 13.7, 5.1 Hz) | | (3.90-3.93) | | |
| 21 | 3.90-3.93 (m, | 58.99 | 3.90-3.93 | 58.99 | 58.99 (2.88, |
| | 1H) | | (2.88) | (3.90-3.93) | 3.06, 8.74) |
| | | | 3.90-3.93 | | |
| | | | (3.06) | | |
| | | | 3.90-3.93 | | |
| | | | (8.74) | | |
| 22 | 8.74 (d, 1H, | | 8.74 | | |
| | J = 6.8 Hz) | | (3.90-3.93) | | |
| 23 | | 150.3 | | | 150.30 |
| | | | | | (7.20-7.26, |
| | | | | | 3.90-3.93) |
| 24 | | 116.5 | | | 116.50 (6.39, |
| | | | | | 6.66, 8.74) |
| 25 | 7.20-7.26 (m, | 134.22 | 7.20-7.26 | 134.22 | 134.22 |
| | 1H) | | (6.39) | (7.20-2.26) | (7.20-7.26) |
| 26 | 6.39 (t, 1H, | 112.48 | 6.39 | 112.48 (6.39) | 112.48 (6.66) |
| | J = 7.5 Hz) | | (7.20-7.26) | | |
| | | | 6.39 | | |
| | | | (7.20-7.26) | | |
| 27 | 7.20-7.26 (m, | 134.48 | 7.20-7.26 | 134.48 | 134.48 |
| | 1H) | | (6.39) | (7.20-7.26) | (7.20-7.26, 6.39) |
| | | | 7.20-7.26 | | |
| | | | (6.66) | | |
| 28 | 6.66 (d, 1H, | 112.63 | 6.66 | 112.63 (6.66) | 112.63 (6.39, |
| | J = 8.6 Hz) | | (7.20-7.26) | | 8.74) |
| 29 | | 195.73 | | | 195.73 |
| | | | | | (7.55-7.58, |
| | | | | | 7.20-7.26) |
| 30 | | 136.8 | | | 136.80 |
| | | | | | (7.28-7.32) |
| 31 (31′) | 7.55-7.58 (m, | 131.25 | 7.55-7.58 | 131.25 | |
| | 2H) | 3JF-C = 8.8 | (7.28-7.32) | (7.55-7.58) | |
| 32 (32′) | 7.28-7.32 (m, | 115.1 | 7.28-7.32 | 115.10 | 115.10 |
| | 2H) | 2JF-C = 21.3 | (7.55-7.58) | (7.28-7.32) | (7.55-7.58) |
| 33 | | 163.4 | | | 163.40 |
| | | 1JF-C = 248 | | | (7.55-7.58, |
| | | | | | 7.28-7.32) |
| 34 | | 173.71 | | | 173.71 (2.88, |
| | | | | | 3.06, 3.90-3.93, |
| | | | | | 8.74) |

FAB-MS (m/z): 595 (M+1)

### Example 4 (−)-2-[(2-(4-fluorobenzoyl)phenyl)amino]-3-[(4-(2-carbazole-ethoxy)phenyl)]propionic acid (Enantiomer 1) Exhibited Activity of PPARα Activator in an In Vitro Reporter Assay Model Experiment

The PPARα reporter gene detection system comprises a pCDNA3.1 luciferase expression plasmid (PPRE enhancer element was inserted into the upstream of the promoter), a green fluorescent protein GFP expression plasmid, and a human PPARα expression plasmid.

The above plasmids were co-transfected (Fugene 6.0 transfection reagent) into human hepatoma cell line L-02. 48 hours after transfection, different concentrations of test compounds were added. 24 hours after administration, the cells were treated with a luciferase assay kit (Promega E1910) and the fluorescence intensity of GFP (wavelength 485-527 nm) and luciferase substrate (wavelength 562 nm) were detected using a fluorescence detector (Fluoroskan Ascent FL) respectively. The GFP signal was used as an internal reference to correct the detected luciferase substrate signal, and a relative reporter activation intensity was obtained by comparing the above corrected signal to the blank control (without treatment of the compounds). The semi-activating activity (EC50) of the test compound was calculated from concentration gradient data.

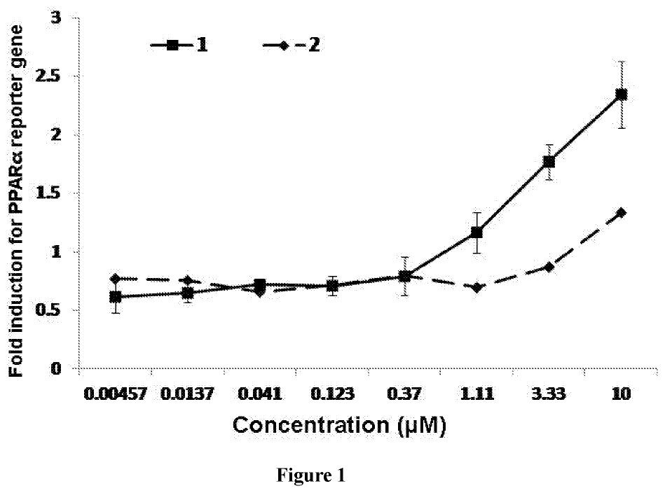

Compared to enantiomer 2, enantiomer 1 has a more significant effect on activating PPARα, with EC₅₀ of 1.135 μM and 5.385 μM, respectively (see FIG. 1).

### Example 5 (−)-2-[(2-(4-fluorobenzoyl)phenyl)amino]-3-[(4-(2-carbazole-ethoxy)phenyl)]propionic acid (Enantiomer 1) Exhibited Activity of RXR/PPARγ Heterodimer Activator in an In Vitro Reporter Assay Model Experiment

The PPARγ reporter gene detection system comprises a pACOX fluorescent reporter gene plasmid (the promoter comprises PPRE sequence), PPARγ expression plasmid, RXR expression plasmid, and a green fluorescent protein GFP expression plasmid.

The above plasmids were co-transfected (Fugene 6.0 transfection reagent) into human hepatoma cell line L-02. 48 hours after transfection, different concentrations of test compounds were added. 24 hours after administration, the cells were treated with a luciferase assay kit (Promega E1910) and the fluorescence intensity of GFP (wavelength 485-527 nm) and luciferase substrate (wavelength 562 nm) were detected using a fluorescence detector (Fluoroskan Ascent FL), respectively. The GFP signal was used as an internal reference to correct the detected luciferase substrate signal, and a relative reporter activation intensity was obtained by comparing the above corrected signal to the blank control (without treatment of the compounds). The semi-activating activity (EC50) of the test compound was calculated from concentration gradient data.

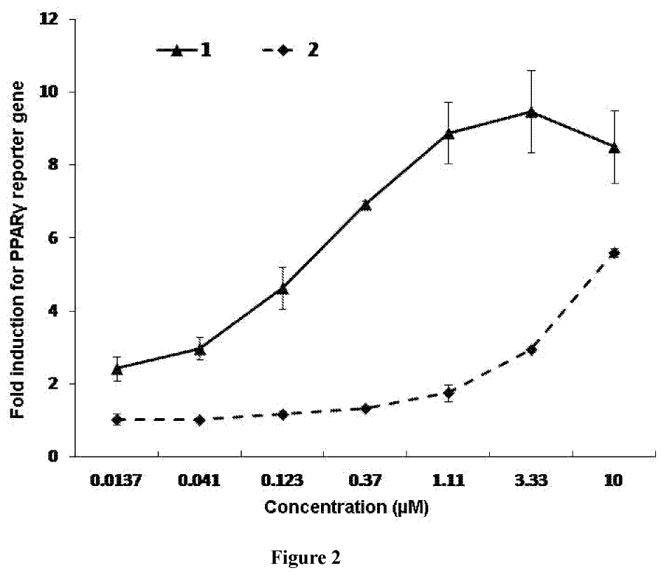

Compared to enantiomer 2, enantiomer 1 has a more significant effect on activating PPARγ, with EC₅₀ of 0.076 μM and 2.709 μM, respectively (see FIG. 2).

### Example 6 (−)-2-[(2-(4-fluorobenzoyl)phenyl)amino]-3-[(4-(2-carbazole-ethoxy)phenyl)]propionic acid (Enantiomer 1) Exhibited Activity of RXR/PPAR-6 Heterodimer Activator in an In Vitro Reporter Assay Model Experiment

The PPARδ reporter gene detection system comprises a pGL3-PPRE fluorescent reporter gene plasmid (PPRE enhancer element was inserted into the upstream of the promoter), PPARδ expression plasmid, RXR expression plasmid, and a green fluorescent protein GFP expression plasmid.

The above plasmids were co-transfected (Fugene 6.0 transfection reagent) into human hepatoma cell line L-02. 48 hours after transfection, different concentrations of test compounds were added. 24 hours after administration, the cells were treated with a luciferase assay kit (Promega E1910) and the fluorescence intensity of GFP (wavelength 485-527 nm) and luciferase substrate (wavelength 562 nm) were detected using a fluorescence detector (Fluoroskan Ascent FL), respectively. The GFP signal is used as an internal reference to correct the detected luciferase substrate signal, and a relative reporter activation intensity was obtained by comparing the above corrected signal to the blank control (without treatment of the compounds). The semi-activating activity (EC₅₀) of the test compound was calculated from concentration gradient data.

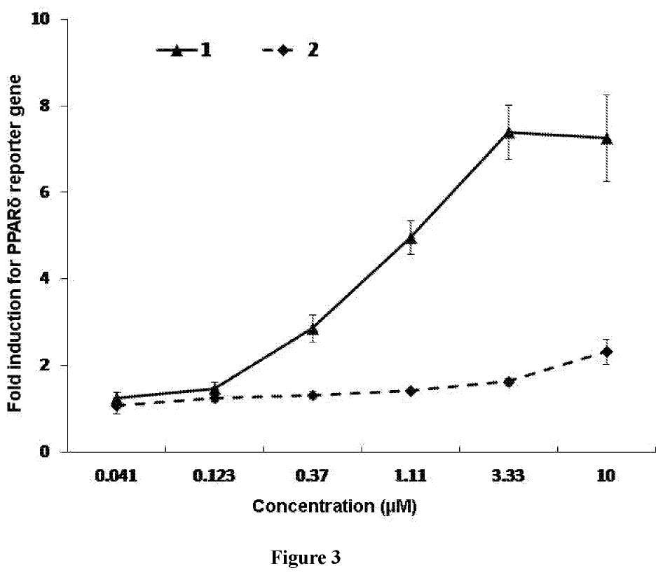

Compared to enantiomer 2, enantiomer 1 has a more significant effect on activating PPAR-6, with EC₅₀ of 1.93 μM and >10 μM (no remarkable activity determined under the highest concentration), respectively (see FIG. 3).

### Example 7 (−)-2-[(2-(4-fluorobenzoyl)phenyl)amino]-3-[(4-(2-carbazole-ethoxy)phenyl)]propionic acid (Enantiomer 1) was Effective in Reducing Blood Glucose Concentrations in Db/Db Mice

The experiment consisted of 4 groups: a model group, two administration groups (administered with enantiomers 1 and 2, respectively) and a positive control group (rosiglitazone), with 10 mice per group. The route of administration was oral, the dose was 20 mg/kg in the administration group and 5 mg/kg in the control group, the frequency of administration was once a day, and the administration period was 14 days. The animals of groups 1 to 4 were orally administered with vehicle, enantiomer 1, enantiomer 2 and the positive drug rosiglitazone, respectively. On days 0, 3, 6, 9, 12, and 14, after fasting for 6 hours after administration, blood was collected from the tail vein and fasting blood glucose levels was measured by a blood glucose meter and recorded. The results are shown in Table 3.

TABLE 3
the effect of (−)-2-[(2-(4-fluorobenzoyl)phenyl)amino]-3-[(4-(2-carbazole-
ethoxy)phenyl)]propionic acid on blood glucose concentrations in mice

| Group | | Day 0 | Day 3 | Day 6 | Day 9 | Day 12 | Day 14 |
|---|---|---|---|---|---|---|---|
| G1: model group (n = 10) | Average value | 18.98 | 25.5 | 23.69 | 26.49 | 29.23 | 18.52 |
| | SEM | 1.47 | 1.12 | 1.17 | 0.97 | 0.99 | 1.28 |
| G2: enantiomer 1(n = 10) | Average value | 18.43 | 18.2 | 14.7 | 18.14 | 16.18 | 10.98 |
| | SEM | 1.35 | 1.87 | 1.33 | 1.42 | 1.48 | 0.83 |
| G3: Enantiomer 2 (n = 10) | Average value | 18.86 | 23.99 | 20.03 | 26.01 | 26.44 | 17.18 |
| | SEM | 1.28 | 1.38 | 1.5 | 1.45 | 1.17 | 1.09 |
| G4: rosiglitazone (n = 10) | Average value | 19.09 | 13.58 | 13.8 | 15.05 | 14.44 | 11.29 |
| | SEM | 1.36 | 1 | 0.82 | 0.86 | 0.46 | 1.09 |

### Example 8 (−)-2-[(2-(4-fluorobenzoyl)phenyl)amino]-3-[(4-(2-carbazole-ethoxy)phenyl)]propionic acid (Enantiomer 1) Increased Glucose Tolerance in the Glucose Tolerance Test (OTGG)

After 14 days of administration, the animals were fasted overnight (<10 hours), and orally given 2 g/kg of glucose on the next day. Blood was collected from the tail vein at the 0 (pre-administration or fasting blood glucose level), 15, 30, 60 and 120 minutes after administration, respectively, and blood glucose levels were then measured by blood glucose meter and recorded. The results are shown in Table 4

TABLE 4
the effect of (−)-2-[(2-(4-fluorobenzoyl)phenyl)amino]-3-[(4-(2-
carbazole-ethoxy)phenyl)] propionic acid on glucose tolerance

| Group | | 0 min | 15 min | 30 min | 60 min | 120 min | AUC |
|---|---|---|---|---|---|---|---|
| G1: model group (n = 10) | Average value | 18.52 | 32.3 | 33.3 | 32.28 | 27.77 | 60.97 |
| | SEM | 1.28 | 0.72 | 0 | 0.58 | 1.96 | 1.54 |
| G2: enantiomer 1(n = 10) | Average value | 10.98 | 28.02 | 32.26 | 26.6 | 14.69 | 47.77 |
| | SEM | 0.83 | 1.34 | 0.78 | 2.15 | 1.13 | 2.33 |
| G3: Enantiomer 2 (n = 10) | Average value | 17.18 | 31.87 | 33.3 | 30.94 | 25.75 | 59.16 |
| | SEM | 1.09 | 1.43 | 0 | 1.3 | 1.8 | 1.59 |
| G4: rosiglitazone (n = 10) | Average value | 11.29 | 28.11 | 31.22 | 25.08 | 13.61 | 45.76 |
| | SEM | 1.09 | 2.03 | 1.11 | 1.33 | 1.48 | 2.01 |

Although the invention has been described in connection with various embodiments, it does not mean that the invention is limited to these embodiments. Those skilled in the art will appreciate that the invention can cover various alternatives, modifications, and equivalents. These alternatives, variations and equivalents are considered to be within the scope of the invention as described herein and the appended claims.

1. (−)-2-[(2-(4-fluorobenzoyl)phenyl)amino]-3-[(4-(2-carbazole-ethoxy)phenyl)]propionic acid or a pharmaceutically acceptable salt thereof.

2. The (−)-2-[(2-(4-fluorobenzoyl)phenyl)amino]-3-[(4-(2-carbazole-ethoxy) phenyl)]propionic acid or a pharmaceutically acceptable salt thereof according to claim 1, wherein the salt is an alkali metal salt, an alkaline earth metal salt, an ammonium salt or a quaternary ammonium salt.

3. The (−)-2-[(2-(4-fluorobenzoyl)phenyl)amino]-3-[(4-(2-carbazole-ethoxy) phenyl)]propionic acid or a pharmaceutically acceptable salt thereof according to claim 2, wherein the alkali metal salt is a sodium or potassium salt, and/or the alkaline earth metal salt is a calcium or magnesium salt.

4. A supercritical chromatography method for preparing the (−)-2-[(2-(4-fluorobenzoyl)phenyl)amino]-3-[(4-(2-carbazole-ethoxy)phenyl)]propionic acid according to claim 1, comprising the steps of:
dissolving 2-[(2-(4-fluorobenzoyl)phenyl)amino]-3-[(4-(2-carbazole-ethoxy)phenyl)] propionic acid in a solvent, loading the solution to a chiral chromatography column, eluting sequentially with a solvent mixture of organic solvent/carbon dioxide and a solvent mixture of organic solvent/carbon dioxide/aqueous ammonia and separating, to obtain (−)-2-[(2-(4-fluorobenzoyl)phenyl)amino]-3-[(4-(2-carbazole-ethoxy)phenyl)]propionic acid.

5. Use of (−)-2-[(2-(4-fluorobenzoyl)phenyl)amino]-3-[(4-(2-carbazole-ethoxy)phenyl)]propionic acid or a pharmaceutically acceptable salt in the preparation of a medicament as a PPAR nuclear receptor activator.

6. Use of (−)-2-[(2-(4-fluorobenzoyl)phenyl)amino]-3-[(4-(2-carbazole-ethoxy) phenyl)]propionic acid or a pharmaceutically acceptable salt in the preparation of a medicament for treating and preventing a disease associated with the regulation of RXR/PPAR.

7. The use according to claim 6, wherein the disease is selected from the group consisting of type 2 diabetes, lipid metabolism disorders, syndrome X, cardiovascular disease, coronary artery disease, hypercholesterolemia and obesity.

8. A pharmaceutical composition, comprising the (−)-2-[(2-(4-fluorobenzoyl)phenyl)amino]-3-[(4-(2-carbazole-ethoxy)phenyl)]propionic acid or a pharmaceutically acceptable salt thereof according to claim 1, and a pharmaceutically acceptable excipient and/or carrier.

9. The pharmaceutical composition according to claim 8, wherein the content of (−)-2-[(2-(4-fluorobenzoyl)phenyl)amino]-3-[(4-(2-carbazole-ethoxy)phenyl)]propionic acid or a pharmaceutically acceptable salt thereof is 0.5-70% by weight.

10. The pharmaceutical composition according to claim 8, wherein said pharmaceutical composition is in the form of an oral formulation, injection preparation or topical preparation.

11. The pharmaceutical composition according to claim 10, wherein the composition is in the form of a tablet, capsule, powder, granule, syrup or pill.
